(19) United States
(12) Patent Application Publication
(10) Pub. No.: US 2019/0223897 A1
(43) Pub. Date: Jul. 25, 2019

(54) SURGICAL INSTRUMENT INCLUDING FLAT AND CURVED HANDLE SURFACES

(71) Applicant: Scanlan International, Inc., St. Paul, MN (US)

(72) Inventors: Mark P. Thuente, St. Paul, MN (US); Rhonda L. Hambleton, Blaine, MN (US); Patrick J. Lowe, Forest Lake, MN (US); Russel T. Scanlan, St. Paul, MN (US)

(21) Appl. No.: 15/880,205

(22) Filed: Jan. 25, 2018

(51) Int. Cl. A61B 17/29

(52) U.S. Cl. CPC A61B 17/2909; A61B 2017/00424

(57) ABSTRACT

An instrument includes a first arm including a first handle and a first tip coupled to the first handle. A second arm is coupled to the first arm, and the second arm includes a second handle and a second tip coupled to the second handle. At least one of the first handle and the second handle includes an inner surface facing toward the other one of the first handle and the second handle. A planar outer surface faces away from the other one of the first handle and the second handle. A first convex curved side surface extends between the inner surface and the planar outer surface. A second convex curved side surface opposite the first convex curved side surface extends between the inner surface and the planar outer surface.

### FIELD OF THE INVENTION

The invention relates generally to surgical instruments. More specifically, the invention relates to surgical instruments including flat and curved handle surfaces to facilitate gripping such instruments.

### BACKGROUND OF THE INVENTION

The handling of delicate tissue during surgical procedures is generally performed utilizing precisely designed surgical instruments. In particular, a surgical forceps (tweezers style), or “pick-ups” is commonly used as it is designed to provide for precisely grasping tissue between the tips. However, grasping such instruments in surgical settings can be difficult, particularly in the presence of one or more fluids (for example, blood of a patient). There remains, therefore, a continuing need for improved gripping structures of surgical instruments.

### SUMMARY OF THE INVENTION

Instruments in accordance with embodiments of the present invention provide an improved grip and facilitate “rolling” such instruments in the hand of a user.

An instrument in accordance with one embodiment of the invention includes a first arm including a first handle and a first tip coupled to the first handle. A second arm is coupled to the first arm, and the second arm includes a second handle and a second tip coupled to the second handle. At least one of the first handle and the second handle includes an inner surface facing toward the other one of the first handle and the second handle. A planar outer surface faces away from the other one of the first handle and the second handle. A first convex curved side surface extends between the inner surface and the planar outer surface. A second convex curved side surface opposite the first convex curved side surface extends between the inner surface and the planar outer surface.

### BRIEF DESCRIPTION OF THE DRAWINGS

FIG. 1A is a top view of a surgical forceps including flat and curved handle surfaces in accordance with an embodiment of the invention.

FIGS. 1B and 1C are side views of the surgical forceps of FIG. 1A illustrating first and second arms thereof in an open position and a closed position, respectively.

FIGS. 2A and 2B are perspective views of a flat and curved handle of an instrument in accordance with an embodiment of the invention.

FIGS. 2C, 2D, and 2E are top, side, and end views, respectively, of the handle of FIGS. 2A and 2B.

FIG. 3 is a partial perspective view of a surgical forceps including flat and curved handle surfaces in accordance with another embodiment of the invention.

### DETAILED DESCRIPTION OF THE DRAWINGS

Instruments in accordance with embodiments of the present invention provide improved tactile characteristics (that is, provide improved “grip”) and facilitate user dexterity. More specifically, instruments in accordance with embodiments of the present invention facilitate “rolling” such instruments in the hand of a user.

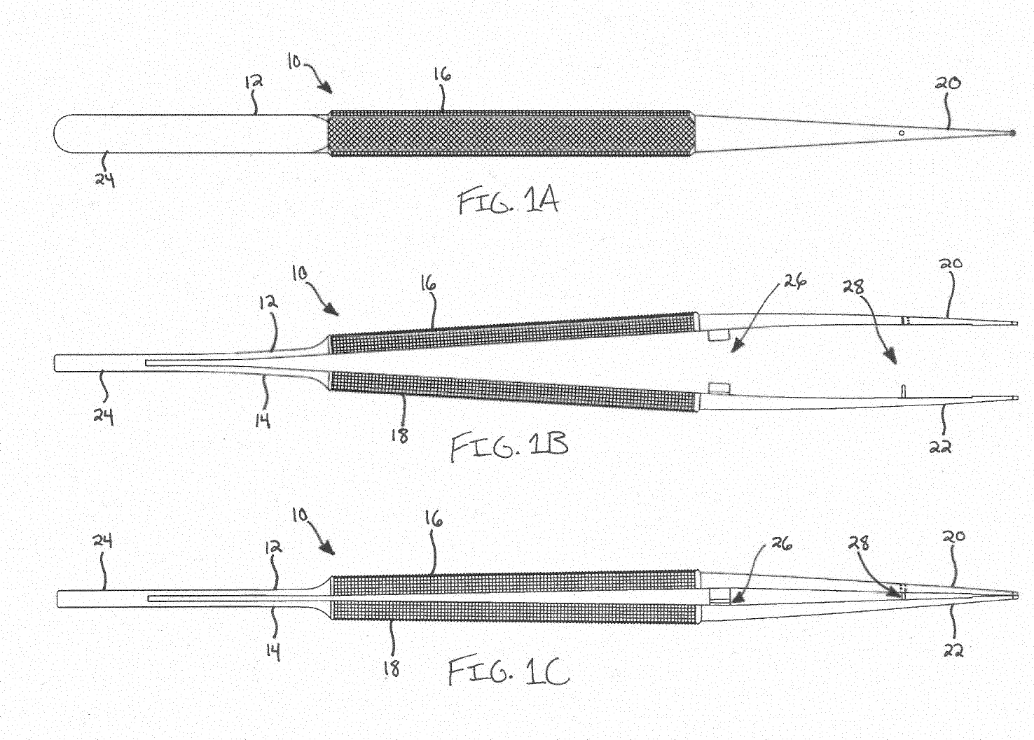

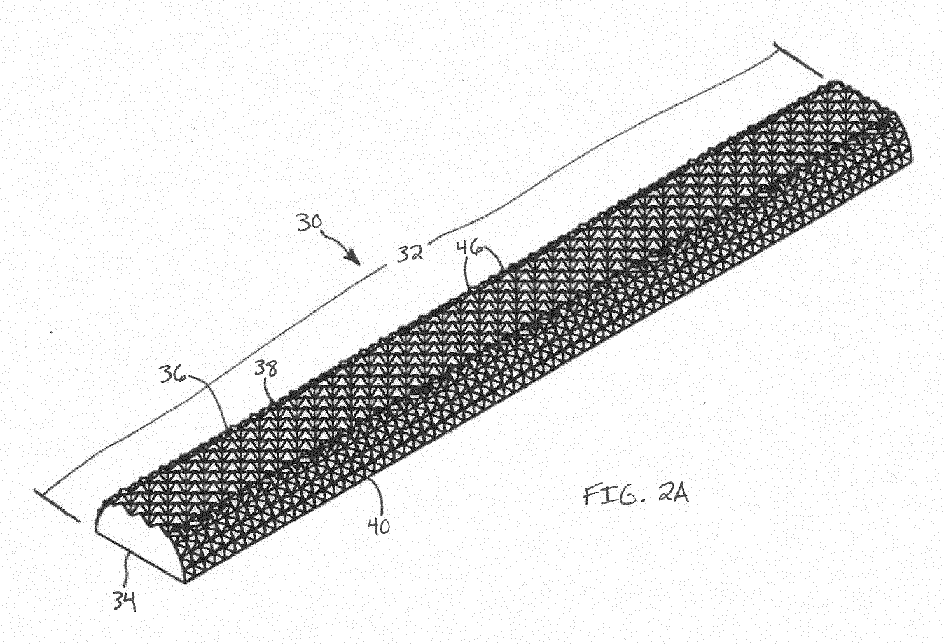

FIGS. 1A-1C illustrate a surgical forceps 10 in accordance with an embodiment of the invention. The forceps 10 includes a first arm 12 and a second arm 14 having a first handle 16 and a second handle 18, respectively. The first handle 16 and the second handle 18 couple to a first tip 20 and a second tip 22, respectively, at the distal ends of the first arm 12 and the second arm 14, respectively. The proximal ends 24 of the first arm 12 and the second arm 14 are joined together in such a manner that the arms 12 and 14 are biased to an open position, as shown in FIG. 1B. During use of the forceps 10 to grasp tissue, a user (more specifically, a surgeon) can hold the arms 12 and 14 at the handles 16 and 18 and squeeze the arms 12 and 14 together against the bias force, thereby moving the arms 12 and 14 to a closed position, as shown in FIG. 1C, in which the tips 20 and 22 may grasp tissue.

In the embodiment shown in FIGS. 1A-1C, the forceps 10 has an overall length (that is, a dimension in a direction extending between the proximal ends 24 and the distal ends of the arms 12 and 14) of substantially 21 cm (that is, 21 cm±10 percent). Other embodiments of forceps have different overall lengths. For example, some embodiments of forceps have an overall length in a range of 18 cm to 25.4 cm.

In the embodiment shown in FIGS. 1A-1C, the arms 12 and 14 couple to a first closing guide 26 and a second closing guide 28 that inhibit crossing of the tips 20 and 22. The first closing guide 26 and the second closing guide 28 may be, for example, any of the closing guides described in U.S. Pat. No. 8,147,512 to Puskas et al., the disclosure of which is hereby incorporated by reference. Other embodiments of forceps lack a first closing guide and/or a second closing guide.

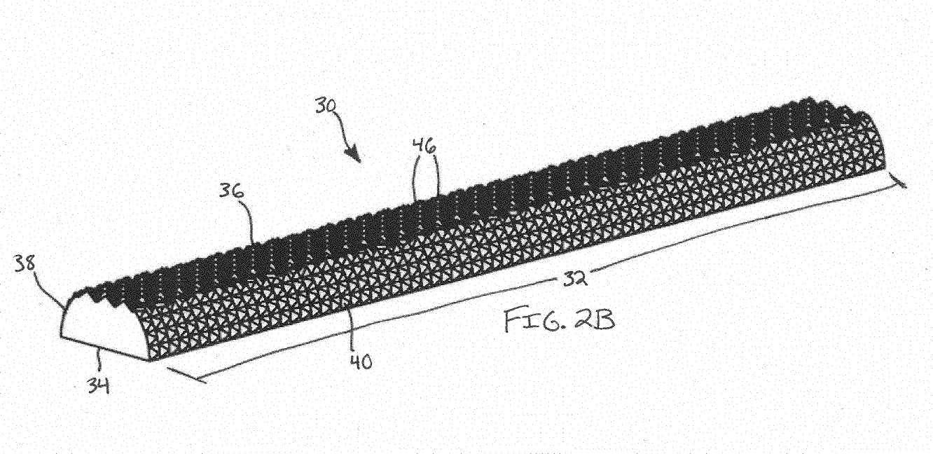

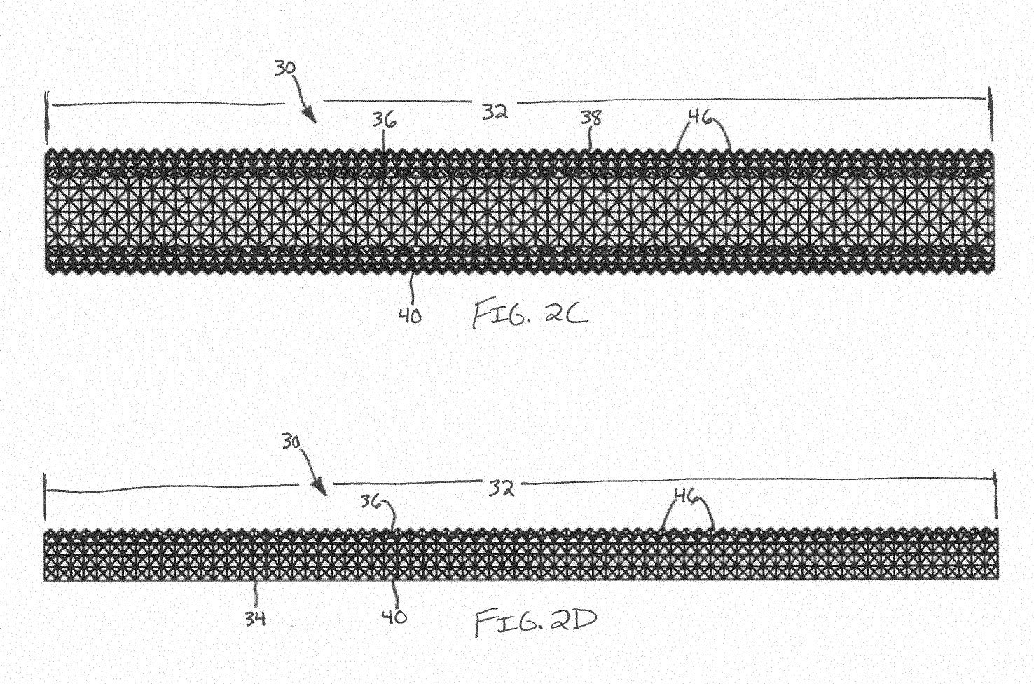

FIGS. 2A-2E illustrate a handle 30 for an instrument in accordance with an embodiment of the invention. The handle 30 may be, for example, the first handle 16 and/or the second handle 18 of the surgical forceps 10 described above. In the illustrated embodiment, the handle 30 is formed of titanium. Other embodiments of handles are formed of one or more different materials, such as stainless steel, aluminum, polyether ether ketone (PEEK), or the like.

In the embodiment shown in FIGS. 2A-2E, the handle 30 has a length 32 (that is, a dimension in a direction extending between the proximal ends 24 and the distal ends of the arms 12 and 14) of substantially 80 mm (that is, 80 mm±10 percent). Other embodiments of handles have different lengths. For example, some embodiments of handles have lengths in a range of 70 mm to 85 mm, more specifically substantially 73 mm (that is, 73 mm±10 percent) or substantially 84 mm (that is, 84 mm±10 percent).

The handle 30 includes a relatively-wide inner surface 34 that faces toward the other handle (for example, the other of the first handle 16 and the second handle 18). Opposite the inner surface 34, the handle 30 includes a relatively-narrow planar outer surface 36 that faces away from the other handle (for example, the other of the first handle 16 and the second handle 18). A first convex curved side surface 38 extends between the inner surface 34 and the outer surface 36. Opposite the first curved side surface 38, a second convex curved side surface 40 extends between the inner surface 34 and the planar outer surface 36.

In the embodiment shown in FIGS. 2A-2E, the handle 30 has a thickness 42 (that is, a dimension in a direction extending between the inner surface 34 and the planar outer surface 36) of substantially 3.9 mm (that is, 3.9 mm±10 percent). In other embodiments, handles have different thicknesses. For example, some embodiments of handles have thicknesses in a range of 3 mm to 5 mm.

In the embodiment shown in FIGS. 2A-2E, the handle 30 has a width 44 (that is, the largest dimension in a direction extending between the first convex curved side surface 38 and the second convex curved side surface 40) of substantially 9.8 mm (that is, 9.8 mm±10 percent). In other embodiments, handles have different widths. For example, some embodiments of handles have widths in a range of 8 mm to 12 mm.

In the embodiment shown in FIGS. 2A-2E, both of the first convex curved side surface 38 and the second convex curved side surface 40 have a uniform radius. More specifically, both of the first convex curved side surface 38 and the second convex curved side surface 40 are circular semi-cylindrical surfaces (that is, a portion of the surface of a circular cylinder or, stated another way, a surface formed by extruding a segment of a circle). Both of the first convex curved side surface 38 and the second convex curved side surface 40 have a radius of substantially 4.9 mm (that is, 4.9 mm±10 percent). In other embodiments of handles, the first convex curved side surface and the second convex curved side surface have a different radius. For example, some embodiments of handles include first convex curved side surfaces and second convex curved side surfaces having a radius in a range of 4 mm to 6 mm.

In other embodiments of handles, one or both of the first convex curved side surface and the second convex curved side surface have a non-uniform or variable radius. More specifically, one or both of the first convex curved side surface and the second convex curved side surface are elliptic semi-cylindrical surfaces (that is, a portion of the surface of an elliptic cylinder or, stated another way, a surface formed by extruding a segment of an ellipse), parabolic semi-cylindrical surfaces (that is, a portion of the surface of an parabolic cylinder or, stated another way, a surface formed by extruding a segment of a parabola), or the like.

In the embodiment shown in FIGS. 2A-2E, the first convex curved side surface 38 and the second convex curved side surface 40 are continuously curved. That is, the first convex curved side surface 38 and the second convex curved side surface 40 lack any planar portions. In other embodiments, one or both of the first convex curved side surface and the second convex curved side surface are discontinuously curved. That is, one or both of the first convex curved side surface and the second convex curved side surface includes one or more planar portions.

In the embodiment shown in FIGS. 2A-2E, each of the planar outer surface 36, the first convex curved side surface 38, and the second convex curved side surface 40 includes a plurality of knurled features 46. The knurled features 46 each have a pyramid shape with a length, width, and height of substantially 1.0 mm (that is, 1.0 mm±10 percent). Stated another way, the knurled features 46 are square diamond knurling. In other embodiments, knurled features include different shapes and/or dimensions. For example, in some embodiments knurled features have frusto-pyramid shapes. As another example, in some embodiments knurled features each have a pyramid shape with a length, width, and height of substantially 1.3 mm (that is, 1.3 mm±10 percent).

In other embodiments of handles, one or more of the planar outer surface, the first convex curved side surface, and the second convex curved side surface lack knurled features. In any case, the planar outer surface 36 is considered to have a planar shape because either (1) the tips of the knurled features 46 are disposed in a common plane, or (2) the surface lacks knurled features and defines a plane. Similarly, the first convex curved side surface 38 and the second convex curved side surface 40 are each considered to have a convex curved shape because either (1) the tips of the knurled features 46 are disposed in a common three-dimensional curve, or (2) the surface lacks knurled features and defines a three-dimensional curve.

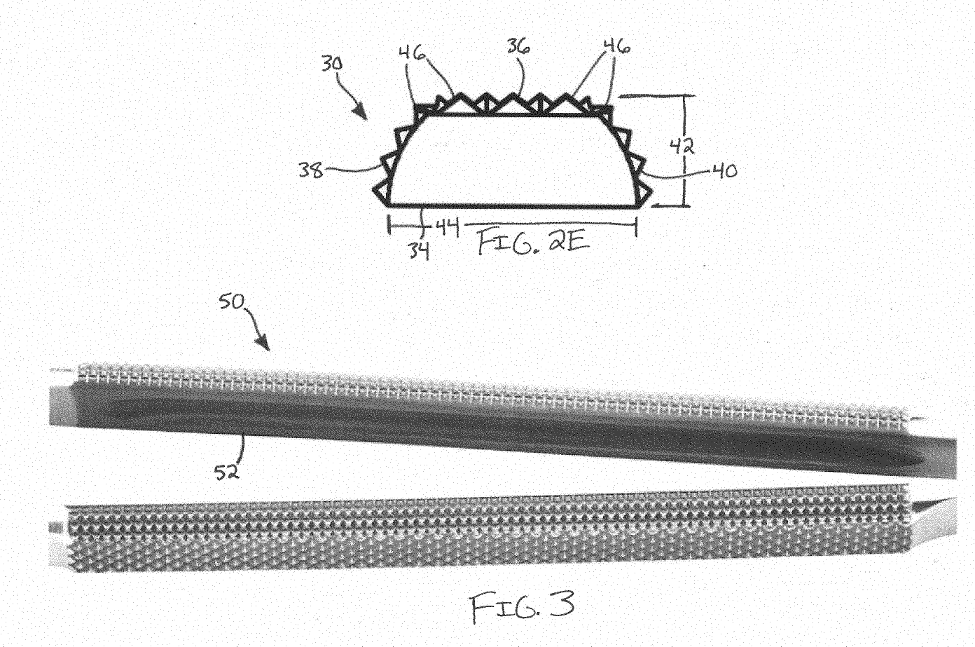

In the embodiment shown in FIGS. 2A-2E, the inner surface 34 has a planar shape. In other embodiments, the inner surface has a different shape. For example and referring to FIG. 3, a handle 50 for an instrument in accordance with an embodiment of the invention is the same as the handle 30, except that an inner surface 52 has a concave shape.

Although the present invention has been described with reference to preferred embodiments, those skilled in the art will recognize that changes can be made in form and detail without departing from the spirit and scope of the invention. In particular, although described in connection with a specific surgical forceps, the flat and convex curved handle surfaces can be incorporated into other surgical instruments having one or more arms handle surfaces, including, for example, other forceps types, needle holders, clamps, clip appliers, bipolar cautery instruments, electro-surgery forceps, and other tweezer-like instruments. The flat and convex curved handle surfaces can also be incorporated into non-medical instruments used in other applications.

What is claimed is:

1. An instrument, comprising:
a first arm comprising a first handle and a first tip coupled to the first handle;
a second arm coupled to the first arm, the second arm comprising a second handle and a second tip coupled to the second handle;
wherein at least one of the first handle and the second handle comprises:
an inner surface facing toward the other one of the first handle and the second handle;
a planar outer surface facing away from the other one of the first handle and the second handle;
a first convex curved side surface extending between the inner surface and the planar outer surface; and
a second convex curved side surface opposite the first convex curved side surface and extending between the inner surface and the planar outer surface.

2. The instrument of claim 1, wherein at least one of the planar outer surface, the first convex curved side surface, and the second convex curved side surface comprises a plurality of knurled features.

3. The instrument of claim 1, wherein each of the planar outer surface, the first convex curved side surface, and the second convex curved side surface comprises a plurality of knurled features.

4. The instrument of claim 1, wherein at least one of the first convex curved side surface and the second convex curved side surface is a semi-cylindrical surface.

5. The instrument of claim 1, wherein each of the first convex curved side surface and the second convex curved side surface are semi-cylindrical surfaces.

6. The instrument of claim 1, wherein at least one of the first convex curved side surface and the second convex curved side surface comprises a radius in a range of 4 mm to 6 mm.

7. The instrument of claim 1, wherein both of the first convex curved side surface and the second convex curved side surface comprise a radius in a range of 4 mm to 6 mm.

8. The instrument of claim 1, wherein at least one of the first convex curved side surface and the second convex curved side surface comprises a radius of substantially 4.9 mm.

9. The instrument of claim 1, wherein both of the first convex curved side surface and the second convex curved side surface comprise a radius of substantially 4.9 mm.

10. The instrument of claim 1, wherein the at least one of the first handle and the second handle has a thickness between the inner surface and the planar outer surface in a range of 3 mm to 5 mm.

11. The instrument of claim 1, wherein the at least one of the first handle and the second handle has a thickness between the inner surface and the planar outer surface of substantially 3.9 mm.

12. An instrument, comprising:
a first arm comprising a first handle and a first tip coupled to the first handle;
a second arm coupled to the first arm, the second arm comprising a second handle and a second tip coupled to the second handle;
wherein the first handle and the second handle each comprise:
an inner surface facing toward the other of the first handle and the second handle;
a planar outer surface facing away from the other of the first handle and the second handle;
a first convex curved side surface extending between the inner surface and the planar outer surface; and
a second convex curved side surface opposite the first convex curved side surface and extending between the inner surface and the planar outer surface.

13. The instrument of claim 12, wherein each of the planar outer surfaces, the first convex curved side surfaces, and the second convex curved side surfaces comprises a plurality of knurled features.

14. The instrument of claim 12, wherein each of the first convex curved side surfaces and the second convex curved side surfaces are semi-cylindrical surfaces.

15. The instrument of claim 12, wherein each of the first convex curved side surfaces and the second convex curved side surfaces comprises a radius in a range of 4 mm to 6 mm.

16. The instrument of claim 12, wherein each of the first convex curved side surfaces and the second convex curved side surfaces comprises a radius of substantially 4.9 mm.

17. The instrument of claim 12, wherein the first handle and the second handle each have a thickness between the inner surface and the planar outer surface in a range of 3 mm to 5 mm.

18. The instrument of claim 12, wherein the first handle and the second handle each have a thickness between the inner surface and the planar outer surface of substantially 3.9 mm.
